(19) United States
(12) Patent Application Publication
(10) Pub. No.: US 2008/0094064 A1
(43) Pub. Date: Apr. 24, 2008

(54) MAGNETIC RESONANCE SYSTEM WITH CIRCULAR GRADIENT SYSTEM AND NON-CIRCULAR WHOLE-BODY COIL

(76) Inventors: Ludwig Eberler, Postbauer-Heng (DE); Wolfgang Renz, Erlangen (DE); Markus Vester, Nurnberg (DE)

Correspondence Address: SCHIFF HARDIN, LLP;PATENT DEPARTMENT, 6600 SEARS TOWER, CHICAGO, IL, 60606-6473, US

(21) Appl. No.: 11/877,850

(22) Filed: Oct. 24, 2007

(30) Foreign Application Priority Data
Oct. 24, 2006 (DE) 10 2006 050 105.5

(51) Int. Cl. G01R 33/32; A61B 5/055

(52) U.S. Cl. 324/318; 600/422

(57) ABSTRACT

A magnetic resonance system has a basic field magnet system that annularly surrounds an examination volume, a gradient system arranged radially within the basic magnetic system, a radio-frequency shield arranged radially within the gradient system and a whole-body coil arranged radially within the radio-frequency shield. The gradient system is essentially circular, such that the gradient system defines a gradient system axis. With regard to a complete circumference around the gradient system axis, the whole-body coil has a first segment and a second segment complementary to the first segment. The first segment covers an angular range of more than 180° relative to the gradient system axis. The whole-body coil exhibits a constant curvature radius in the first segment and is more gently curved in the second segment than in the first segment. The whole-body coil is arranged in the gradient system such that a radio-frequency field return that is possible in the center of the first segment and a radio-frequency field return that is possible in the center of the second segment are at least approximately equal.

### BACKGROUND OF THE INVENTION

1. Field of the Invention

The present invention concerns a magnetic resonance system.

2. Description of the Prior Art

The basic components of magnetic resonance systems are generally known. A basic magnetic system is present that annularly surrounds an examination volume. A gradient system is arranged radially within the basic magnetic system. A radio-frequency shield is arranged radially within the gradient system. A whole-body coil is arranged radially within the radio-frequency shield. The gradient system is normally essentially circular, such that the gradient system defines a gradient system axis.

In magnetic resonance system it is attempted to dimension the various magnet systems (basic magnetic system, gradient system, whole-body coil) as small as possible while still achieving an optimally large opening for the patient tunnel. To achieve this goal in the prior art, the basic magnetic system, the gradient system and the whole-body coil are normally fashioned similarly—for example circularly or elliptically—and concentrically nested within one another.

A magnetic resonance system in which a gradient system is arranged radially within a basic magnetic system is known from “A high-efficiency asymmetric Gradient Coil” by J. A. Overweg and J. Weizenecker, appearing in Proc. Intl. Soc. Mag. Reson. Med. 11 (2003), page 744. The gradient system exhibits a cross-section that has two segments complementary to one another. One segment is fashioned as a circular arc that exhibits a first radius and an angular coverage of more than 180° relative to the center point of the circular arc. The second segment is likewise fashioned as a circular arc, but it exhibits a significantly larger radius of curvature and a significantly smaller angular coverage relative to its center point.

A magnetic resonance system is known from WO 2005/050237 A1 in which a basic magnetic system annularly surrounds an examination volume. Arranged radially inside the basic magnetic system is a gradient system, arranged radially inside the gradient system is a radio-frequency shield, and arranged radially inside the radio-frequency shield is a whole-body coil. Relative to its complete circumference, the whole-body coil exhibits a first segment and a second segment complementary to the first segment. In the first segment the whole-body coil exhibits a constant curvature radius. The constant curvature radius defines one axis of the whole-body coil. The first segment covers an angular range of approximately 180° relative to this axis. The second segment is more gently curved than the first segment.

A similar disclosure is present in United States Patent Application Publication No. 2005/127913 A1, but in the magnetic resonance system disclosed in there the first segment covers an angular range of more than 180°.

A similar magnetic resonance system is known from U.S. Pat. No. 4,686,473. In this magnetic resonance system the whole-body coil has only a first segment that exhibits approximately the shape of a half ellipse.

### SUMMARY OF THE INVENTION

An object of the present invention is to provide a magnetic resonance system that enables a large opening for a patient tunnel with a cost-effective and simple design.

The object is achieved by a magnetic resonance system according to the invention having a basic magnetic system that annularly surrounds an examination volume, a gradient system arranged radially within the basic magnetic system, a radio-frequency shield arranged radially within the gradient system, and a whole-body coil is arranged radially within the radio-frequency shield. The gradient system is essentially circular, such that the gradient system defines one gradient system axis. With regard to a complete circumference around the gradient system axis, the whole-body coil has a first segment and a second segment complementary to the first segment. The first segment covers an angular range of more than 180° relative to the gradient system axis. The whole-body coil exhibits a constant radius of curvature in the first segment and is more gently curved in the second segment than in the first segment. The whole-body coil is arranged in the gradient system such that a radio-frequency field return possible in the center of the first segment and a radio-frequency field return possible in the center of the second segment that are at least approximately equal in size.

The efficiency of the whole-body coil can be optimized by this structure. In particular an amplification of the horizontal magnetic field polarization relative to the vertical magnetic field polarization results in this structure due to the significantly lower lateral distance. Due to the typical cross-section of examination patients, this is advantageous for the power requirement that is necessary in order to achieve a desired excitation flip angle in a patient. In data acquisition mode, this procedure enables a somewhat higher signal-to-noise ratio than in the case of a symmetrical circular polarization.

The radio-frequency shield preferably radially adjoins the inside of the gradient system. The embodiment of the magnetic resonance system can be optimized even further thereby.

There are a number of possible embodiments of the second segment. For example, it is possible for the second segment to be fashioned as a straight segment or as an arced segment. However, it is preferable that the second segment is fashioned as a smooth curve with non-constant curvature, for example as an ellipse or as an oval. With the last cited embodiment it is possible for the first segment and the second segment to abut one another tangentially.

The first segment is preferably fashioned as a shell segment of a cylinder that, viewed transverse to the gradient system axis, covers a circular arc of at least 180°. This allows the patient space above a typically present patient bed to be essentially unchanged. The same “look and feel” therefore results for the patient as for a magnetic resonance system of the prior art.

The whole-body coil normally has ferrules extending around the gradient system axis and antenna rods connecting the ferrules, whereby the antenna rods are arranged distributed around the gradient system axis. A whole-body coil is fashioned in this manner, as is conventional, is known as a birdcage resonator. With such a birdcage resonator the field distribution can be optimized by the antenna rods being arranged approximately equidistantly per segment in the first segment and in the second segment, relative to the arc length.

### BRIEF DESCRIPTION OF THE DRAWINGS

FIG. 1 schematically illustrates a magnetic resonance system from the side, partially in section.

FIG. 2 is a section through the magnetic resonance system of FIG. 1.

FIGS. 3 and 4 show alternative embodiments of the magnetic resonance system of FIGS. 1 and 2.

FIG. 5 is a schematic perspective representation of a typical whole-body coil.

FIG. 6 is a section through the whole-body coil of the system of FIG. 1.

### DESCRIPTION OF THE PREFERRED EMBODIMENTS

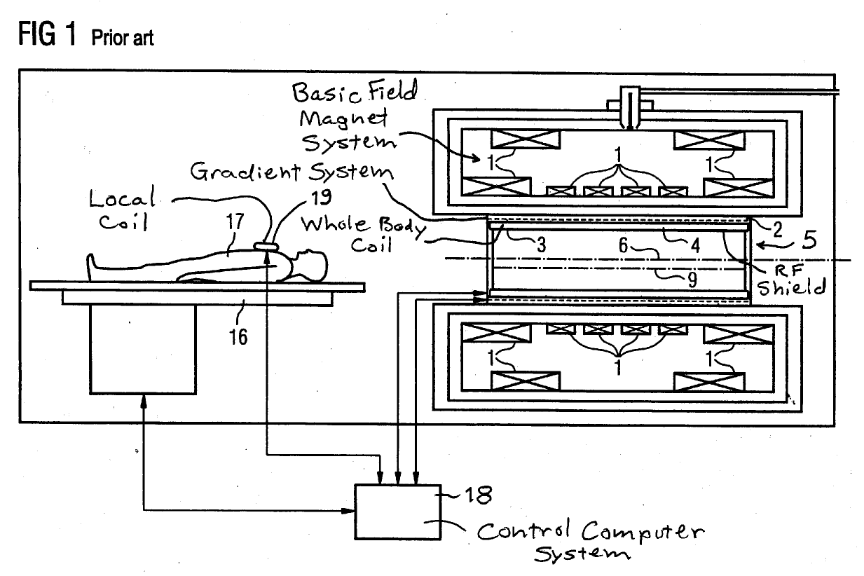

As shown in FIGS. 1 and 2 a magnetic resonance system has a gradient magnet system 1, a gradient system 2, a radio-frequency shield 3 and a whole-body coil 4. The basic field magnet system 1 annularly surrounds an examination volume 5, normally circularly or at least essentially circularly. The gradient system 2 is radially arranged within the basic field magnet system 1. It is likewise normally fashioned circularly or at least essentially circularly. It is normally arranged concentrically relative to the basic field magnet system 1.

Due to its at least essentially circular design, the gradient system 2 defines a gradient system axis 6 around which the basic field magnet system 1 and the gradient system 2 turn. The term “radial” therefore relates to the gradient system axis 6. It designates a direction perpendicular to the gradient system axis 6, toward the gradient system axis 6 or away from it. The term “tangential” likewise designates a direction perpendicular to the gradient system axis 6, bur not toward or away from the gradient system axis 6, but rather around it. The term “axial” designates a direction parallel to the gradient system axis 6.

The gradient system 2 has at least one gradient coil. Actually, three gradient coils are normally present for the three directions of a Cartesian coordinate system. Such gradient coils are known and are not shown in figures.

The radio-frequency shield 3 is arranged radially within the gradient system 2. As can be seen from FIG. 1, it normally radially adjoins the inside of the gradient system 2.

A local coil 19 may be additionally provided.

All components are operated in a known manner by a control computer system 18.

The whole-body coil 4 is arranged radially within the radio-frequency shield 3. in accordance with the invention, as shown in FIG. 2, it has a first segment 7 and a second segment 8 relative to one complete circumference around the gradient system axis 6. The second segment 8 is complementary to the first segment 7, meaning that the first and second segments 7, 8 together produces the one complete circumference around the gradient system axis 6. The whole-body coil 4 is normally provided with a radial internal cladding. Depending on the embodiment of the magnetic resonance system, the cladding can at the same time be a support structure for the whole-body coil 4.

The first segment 7 covers (encompasses) an angle range a of more than 180° relative to the gradient system axis 6. In the first segment 7 the whole-body coil 4 exhibits a constant curvature radius r. The first segment 7 therefore defines a whole-body axis 9.

Due to its constant curvature radius r, the first segment 7 is fashioned as a shell segment of a cylinder. Viewed in cross-section transverse to the gradient system axis 6, it covers a circular arc. Relative to the whole-body axis 9, the circular arc exhibits a coverage angle β that, according to FIG. 2, is exactly 180°. However, the coverage angle β could alternatively be less than or (advantageously) greater than 180°.

In the second segment 8 the whole-body coil 4 is more gently curved than in the first segment 7. According to FIG. 2, the second segment 8 is fashioned, for example, as a smooth curve that exhibits a non-constant curvature. The second segment 8 can in particular be fashioned as an ellipse or as an oval.

The first segment 7 exhibits a center 10, the second segment 8 a center 11. The center 10 of the first segment 7 exhibits a first distance a from the gradient system 2. The center 11 of the second segment 8 exhibits a second distance b from the gradient system 2. The distances a, b are approximately equal in size. It is thus made possible for a radio-frequency field return in the center 10 of the first segment 7, and a radio-frequency field return in the center 11 of the second segment 8, to be at least approximately equal in size.

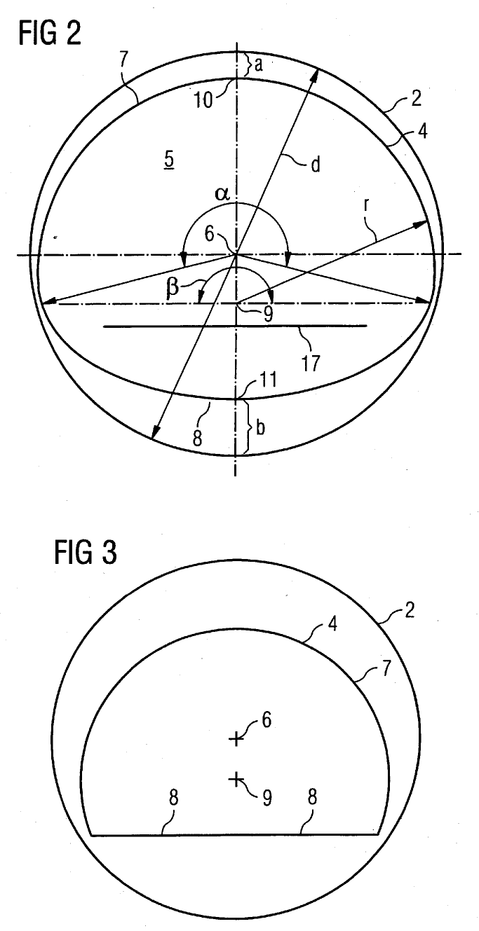

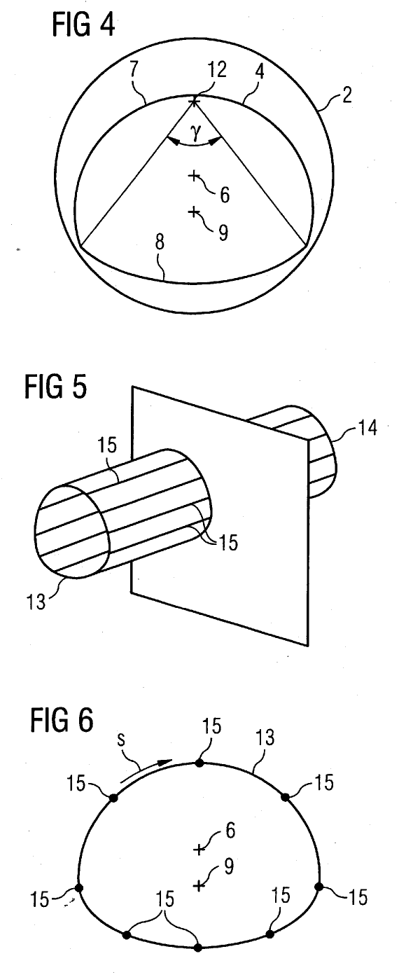

As shown in FIG. 2, the first and second segments 7, 8 tangentially adjoin one another. This embodiment is preferable but not necessary. This is subsequently explained in connection with FIGS. 3 and 4.

The embodiments of FIGS. 3 and 4 essentially correspond to the embodiment of FIG. 2. Therefore only the differences are discussed. The significant differences of FIGS. 3 and 4 relative to FIG. 2 are the size of the coverage angle β of the circular arc of the first segment 7 and the embodiment of the second segment 8.

According to FIG. 3, the second segment 8 is fashioned straight. In the alternative embodiment of FIG. 4 the second segment 8 is fashioned as a circular arc around an arc center point 12. In contrast to the first segment 7, a coverage angle γ of the second segment 8 relative to the arc center point 12 is, however, smaller than 180°, normally significantly smaller.

The whole-body coil 4 is normally fashioned as a birdcage resonator according to FIGS. 5 and 6. As is typical, the birdcage resonator 4 comprises two ferrules 13, 14 as well as antenna rods 15. Corresponding to the embodiment of the whole-body coil 4, the ferrules 13, 14 extend around the gradient system axis 6. The antenna rods 15 connect the ferrules 13, 14 with one another. They are arranged distributed around the gradient system axis 6.

Given a conventional embodiment of the magnetic resonance system, the ferrules 13, 14 are normally fashioned in a circular or elliptical manner and are fashioned concentric relative to the gradient system axis 6. Given the conventional embodiment of the magnetic resonance system, the antenna rods 15 can therefore be arranged uniformly distributed from one another both with regard to an arc length and with regard to an angle segment of the antenna rods 15. In contrast to this, in the inventive embodiment the ferrules 13, 14 are neither fashioned circularly nor arranged symmetrical to the gradient system axis 6 or the whole-body axis 9. A uniform distribution of the antenna rods 15 can therefore at maximum be achieved with regard to a single one of the criteria: arc length s—angle distance from antenna rod 15 to antenna rod 15, relative to the gradient system axis 6—angle distance from antenna rod 15 to antenna rod 15, relative to the whole-body axis 9. In the most general case, a uniform distribution of the antenna rods 15 exists with regard to more than one of the criteria.

As shown in FIG. 6, the antenna rods 15 are arranged at least approximately equidistantly relative to the arc length s within the first segment 7 and within the second segment 8. Independently of whether the distance of the antenna rods 15 in the first segment 7 is equal to the distance of the antenna rods 15 in the second segment 8 or not, however, the distribution of the antenna rods 15 should be selected such that the resulting magnetic resonance-active radio-frequency field component is at least approximately homogeneous. With a suitable distribution of capacitances on the antenna rods 15 and the ferrules 13, 14, it is even possible to bring a number of modes of the birdcage resonator 4 to this same resonance frequency. As in the prior art, in this embodiment it is possible to operate the whole-body coil 4 as an array antenna with a number of independently-fed sub-antennas.

In comparison to a conventionally designed magnetic resonance system, in the inventive embodiment of the magnetic resonance system the space that is available to a patient 17 situated on a patient bed 16 can be maintained without modification. At the same time the inner diameter d of the gradient system 2 can be reduced by approximately 5 to 10% without impairing the quality of the radio-frequency field of the whole-body coil 4. Nevertheless, the inventively designed magnetic resonance system can be produced simply and cost-effectively.

Although modifications and changes may be suggested by those skilled in the art, it is the intention of the inventors to embody within the patent warranted hereon all changes and modifications as reasonably and properly come within the scope of their contribution to the art.

We claim as our invention:

1. A magnetic resonance system comprising:
a basic magnet system that annularly surrounds an examination volume;
a gradient coil system disposed radially within the basic magnet system;
a radio-frequency shield disposed radially within the gradient coil system;
a whole-body coil disposed radially within the radio-frequency shield;
said gradient coil system having a circular configuration and thereby defining a gradient coil axis; and
within one complete circumference around said gradient coil system axis, said whole body coil consisting of a first segment and a second segment complementary to the first segment, said first segment encompassing an angular range of more than 180° relative to said gradient coil system axis and said whole body coil having a constant radius of curvature in said first segment and being less curved in said second segment than in said first segment, and said whole body coil being disposed in said gradient system making a radio-frequency field return in a center of said first segment and a radio-frequency field return in the center of the second segment substantially equal.

2. A magnetic resonance system as claimed in claim 1 wherein said radio-frequency shield radially abuts an interior of said gradient coil system.

3. A magnetic resonance system as claimed in claim 1 wherein said second segment is a straight segment.

4. A magnetic resonance system as claimed in claim 1 wherein said second segment is circular arced.

5. A magnetic resonance system as claimed in claim 1 wherein said second segment is smoothly curved with a non-constant curvature.

6. A magnetic resonance system as claimed in claim 5 wherein said second segment exhibits a shape selected from the group consisting of an ellipse and an oval.

7. A magnetic resonance system as claimed in claim 5 wherein said first and second segments tangentially abut each other.

8. A magnetic resonance system as claimed in claim 1 wherein said first segment is a shell segment of a cylinder that, in a cross-section perpendicular to the gradient coil system axis, encompasses a circular arc of at least 180°.

9. A magnetic resonance system as claimed in claim 1 wherein said whole body coil comprises end ferrules extending around said gradient coil system axis, and a plurality of antenna rods connected between the end ferrules, said antenna rods being distributed around the gradient coil system axis.

10. A magnetic resonance system as claimed in claim 9 wherein said antenna rods are equidistant from each other with regard to an arc length between neighboring antenna rods along said first and second segments.
